(19) United States
(12) Patent Application Publication
(10) Pub. No.: US 2024/0269408 A1
(43) Pub. Date: Aug. 15, 2024

(54) SYSTEMS AND METHODS FOR SUPPLEMENTAL OXYGEN DELIVERY

(71) Applicant: Ravit BARKAMA, Tenafly, NJ (US)

(72) Inventor: Ravit BARKAMA, Tenafly, NJ (US)

(21) Appl. No.: 18/562,442

(22) PCT Filed: May 19, 2022
(86) PCT No.: PCT/IL2022/050520

§ 371 Date: Nov. 20, 2023

Related U.S. Application Data
(60) Provisional application No. 63/190,875, filed on May 20, 2021.

(51) Int. Cl. A61M 16/00; A61M 16/10

(52) U.S. Cl. CPC A61M 16/024; A61M 2016/1025; A61M 2202/0208; A61M 2205/3327; A61M 2205/3546

(57) ABSTRACT

A system and method for monitoring a subject receiving supplemental oxygen and controlling supplemental oxygen delivery thereto, which includes utilizing one or more sensors to monitor physiologic parameters of the subject, analyzing the monitored physiologic parameters, and subject specific data and/or population data, to determine whether modification of treatment parameters, and/or attention of a caregiver, is required, and automatically modifying one or more of the treatment parameters or alerting the caregiver and/or subject, such that the treatment parameters of the subject may be modified to optimize the supplemental oxygen treatment. The method and system provided herein facilitate personalization of oxygen delivery to a subject in a need thereof.

### TECHNICAL FIELD

The present disclosure relates generally to supplemental oxygen delivery systems and methods.

### BACKGROUND

Supplemental oxygen therapy is widely available and is one of the more commonly prescribed interventions. When administered correctly supplemental oxygen may be life-saving, however, oxygen is often given in a suboptimal manner. Inappropriate dose, inadequate method of delivery, and failure to monitor treatment may have serious consequences on the treated subject's health. In a recent hospital survey, about 85% of subjects receiving oxygen were inadequately supervised and therefore sub-optimally treated. Hyperoxia, for example, may cause complex effects on several physiologic functions. It may affect alveolar ventilation or perfusion, may reverse hypoxic vasoconstriction, may induce pulmonary toxicity, and may reduce tissue blood flow due to vasoconstriction.

Better monitoring of patient status, the ability to perform early diagnosis of deterioration, understanding if the new condition could benefit from intervention or modification of treatment parameters and defining which intervention would be optimal, could all potentially improve patient outcomes.

### SUMMARY

The following embodiments and aspects thereof are described and illustrated in conjunction with systems, tools and methods which are meant to be exemplary and illustrative, not limiting in scope.

According to some embodiments of the present invention there is provided a computerized method for monitoring a subject receiving supplemental oxygen and/or being in risk of requiring supplemental oxygen, and controlling supplemental oxygen delivery thereto, the method including operations of: utilizing one or more sensors to monitor physiologic parameters of the subject, the physiologic parameters including at least oxygen saturation (SaO₂) level and one or more of respiratory rate and heart rate, utilizing a processing circuitry to analyze the monitored physiologic parameters, and subject specific data and/or population data, to determine whether (i) modification of supplemental oxygen delivery treatment parameters/initiation of treatment with supplemental oxygen, and/or (ii) attention of a caregiver, is required, and if at least (i) is required: automatically modifying one or more of the supplemental oxygen delivery treatment parameters, and if at least (ii) is required: alerting the caregiver and/or subject, and/or providing thereto recommended modified treatment parameters, wherein the supplemental oxygen delivery treatment parameters include a supplemented oxygen flow rate and type of oxygen delivery device, a fraction of inspired oxygen (FiO₂), and wherein the population data include treatment data of a plurality of subjects receiving supplemental oxygen and in a same medical subgroup as the subject.

Advantageously, the method and system provided herein facilitate personalization of oxygen delivery to a subject in a need thereof.

According to some embodiments, the SaO₂ level referred to herein, is peripheral oxygen saturation (SpO₂), which is an estimation of the oxygen saturation level typically measured with a pulse oximeter device.

According to some embodiments, automatically modifying one or more of the supplemental oxygen delivery treatment parameters may include initiating supplemental oxygen delivery treatment.

According to some embodiments, the method includes identifying the currently used oxygen delivery device.

According to some embodiments, the method includes identifying that a current (or currently used) oxygen delivery device and/or mode is suboptimal and/or identifying that the subject needs initiation of treatment with supplemental oxygen.

According to some embodiments, the analysis operation includes: (a) computing a target SaO₂ level, and contingent on a current SaO₂ level being sufficiently lower than the target SaO₂ level, and, optionally, further assessed not to increase under the current treatment to sufficiently near the target SaO₂ level: (b) computing adjusted values of the treatment parameters, which are configured to raise the current SaO₂ level to the target SaO₂ level.

According to some embodiments, contingent on (b), in the operation of modifying the treatment parameters, if implemented, the treatment parameters are modified to the adjusted values, and in the operation of providing the recommendation, if implemented, the adjusted values are recommended.

According to some embodiments, one or more of the automatically modifiable treatment parameters are limited to modifications to within a treatment range, and modifications to outside the treatment range require intervention of caregiver.

According to some embodiments, the treatment range of an automatically modifiable treatment parameter is population and/or subject-specific.

According to some embodiments, the recommended values of at least some of the treatment parameters are limited to within a respective safety range.

According to some embodiments, in the monitoring operation, the physiologic parameters are monitored continuously or semi-continuously.

According to some embodiments, in the monitoring operation, the physiologic parameters are repeatedly sampled at a rate correlating with a level of severity of the condition of the subject.

According to some embodiments, in the analyzing operation, one or more algorithms, generated based on machine learning and/or data mining tools, are employed.

According to some embodiments, the method further includes an initial training operation wherein the one or more algorithms are generated taking into account a set of training examples extracted at least from the population data.

According to some embodiments, the one or more algorithms include an artificial neural network and/or a decision tree.

According to some embodiments, the plurality of physiologic parameters further includes one or more of blood O₂, blood CO₂, exhaled CO₂, and oxygen reserve index.

According to some embodiments, the subject is further monitored for one or more additional physiological parameters other than respiratory parameters, wherein in the operation of analyzing the monitored respiratory parameters one or more additional physiological parameters are taken into account, and wherein the one or more physiological parameters include a temperature, blood pressure, and/or blood sugar level of the subject.

According to some embodiments, the respiratory infection is viral and/or bacterial and/or mycobacterium and/or fungal and/or aspiration pneumonia wherein the respiratory infection is manifested at least as pneumonia.

According to some embodiments, the respiratory infection is COVID-19 and/or pneumococcal and/or tuberculosis.

According to some embodiments, the subject specific data further includes one or more of at least a partial medical history of the subject and a response of the subject to past treatments.

According to some embodiments, the medical subgroup of subjects is characterized by a same gender, age bracket, ethnicity, weight category as the subject are diagnosed with a same or a similar medical condition as the subject, medical comorbidities, cardiac and respiratory conditions, and/or similar treatments for their chronic conditions.

According to some embodiments, the treatment parameters further include intervals of oxygen supplementation, timing of oxygen supplementation

According to some embodiments, the delivery mode is nasal oral, oral-nasal, and/or by tracheostomy.

According to some embodiments, the treatment parameters further specify, per each physical delivery mode, one or more types of facial devices compatible with the delivery mode.

According to some embodiments, the types of facial devices include any one or more of a nasal cannula, an oral-nasal oxygen mask, a non-rebreather mask, high flow nasal cannula, high flow oxygen delivery devices, and a full face oxygen mask.

According to some embodiments, the sensors include one or more of a pulse oximeter, respiratory rate, a heart-rate sensor, a FiO₂ sensor, a capnograph, an ECG sensor, a blood pressure sensor, a thermometer, a galvanic skin response sensor, a motion sensor, activity sensor and pedometer.

According to some embodiments, the method may further include defining normal or predefined ranges of the supplemented oxygen flow rate and/or the fraction of inspired oxygen (FiO₂), monitoring a trend in the supplemented oxygen flow rate and/or the fraction of inspired oxygen (FiO₂) during treatment, and if a persistent trend (increase or decrease) is detected, activating a respective alarm indicative of a deterioration or an improvement in the subject's condition.

According to some embodiments, the method may further include providing a recommendation to the subject, the recommendation comprises: a number of hours per day the subject is required to receive supplemental oxygen treatment, a duration of a period in which the subject can take a break from supplemental oxygen treatment and/or from one or more sensors (of course, the subject may choose to reconnect to the supplemental oxygen treatment/sensor(s) should they experience a deterioration), indication of a time, when the subject is required to provide physiological parameters, or any combination thereof.

According to some embodiments, the method may further include assessing the subject's physical activity, for example, utilizing a motion sensor, activity sensor and/or pedometer. Depending on the results of the assessment, the controller may suggest a consciousness level assessment. Depression of consciousness may suggest CO₂ narcosis. Advantageously, physical activity assessment may assist in determining when hypoxemia is real as opposed to an artifact due to patient movement or weak pulse oximeter signal. Moreover, according to some embodiments, if the system detects that the subject engages in physical activity (e.g., sports, climbing stairs, walking, etc.), the controller may further recommend or instruct to automatically provide more O₂ to the subject in order to prevent exertional hypoxemia.

According to some embodiments, the method may further include recommending a “challenge test”, such as a few (e.g., 3-6) minutes walk or climbing stairs, and predicting an upcoming oxygen desaturation, based at least on a result of the challenge test.

According to some embodiments of the present invention there is provided a system for regulating delivery of supplemented oxygen to a subject receiving oxygen therapy and/or being in risk of requiring supplemental oxygen, the system including a controller and communication infrastructure configured to communicatively associate the controller with (i) an electronic valve, which is fluidly coupled to an oxygen source and configured to control a flow of supplemental oxygen, (ii) a plurality of sensors monitoring physiologic parameters of the subject, wherein the controller is configured to: receive values of sensor-obtained physiologic parameters of the subject, analyze the values and subject specific data and/or population data to determine whether (i) modification of supplemental oxygen delivery treatment parameters/initiation of treatment with supplemental oxygen, and/or (ii) attention of a caregiver and/or subject, is required, and if at least (i) is required: instruct the electronic valve to change the flow of supplemental oxygen, such as to modify one or more of the supplemental oxygen delivery treatment parameters, and if at least (ii) is required: alert the caregiver and/or provide thereto recommended modified supplemental oxygen delivery treatment parameters, wherein the physiologic parameters include at least oxygen saturation (SaO₂) level and one or more of respiratory rate and heart rate, wherein the supplemental oxygen delivery treatment parameters include a supplemented oxygen flow rate and a fraction of inspired oxygen, and wherein the population data include treatment data of a plurality of subjects receiving supplemental oxygen and in a same medical subgroup as the subject. According to additional or alternative embodiments, the system may be configured to identify a subject at risk for requiring supplemental oxygen, and/or identifies oxygenation issues. The system may further alert that the patient is deteriorating, and initiation of supplemental oxygen is needed.

According to some embodiments, the controller is configured to identify the currently used oxygen delivery device.

According to some embodiments, the controller is configured to identify that a current (or currently used) oxygen delivery device and/or mode is suboptimal and/or identifying that the subject needs initiation of treatment with supplemental oxygen.

According to some embodiments, change the flow of supplemental oxygen may include initiate supplemental oxygen delivery.

According to some embodiments, the analysis performed by the controller includes: (a) computing a target SaO₂ level, and contingent on a current SaO₂ level being sufficiently lower than the target SaO₂ level, and, optionally, further assessed not to increase under the current treatment to sufficiently near the target SaO₂ level: (b) computing adjusted values of the treatment parameters, which are configured to raise the current SaO₂ level to the target SaO₂ level.

According to some embodiments, and contingent on (b), and (i), the controller is configured to modify the one or more treatment parameters to the respective adjusted values, and wherein, contingent on (b), and (ii), the controller is configured to recommend the adjusted values.

According to some embodiments, the controller is configured such that one or more of treatment parameters automatically modifiable by the system are limited to modifications to within a treatment range, and modifications to outside the treatment range require authorization by a caregiver.

According to some embodiments, the system further includes the electronic valve.

According to some embodiments, the system further includes a console housing the controller, the electronic valve, and at least part of the communication infrastructure, and wherein the console includes an inlet port and an outlet port, each of which is fluidly-coupled to the electronic valve, the inlet port being configured to be connected to a first tube so as to fluidly couple the electronic valve to the oxygen source, the outlet port being configured to be connected to a second tube so as to fluidly couple the electronic valve to a facial device for supplemental oxygen delivery.

According to some embodiments, the system is portable and/or for home use.

According to some embodiments, the facial device is a nasal cannula, an oral-nasal oxygen mask, a non-rebreather mask, high flow nasal cannula, high flow oxygen delivery devices, a full face oxygen mask.

According to some embodiments, the respiratory parameters further include one or more of blood O₂, blood CO₂, exhaled CO₂, and oxygen reserve index.

According to some embodiments, the controller is configured to identify a delivery mode of the supplemental oxygen.

According to some embodiments, the treatment parameters further include a delivery mode of the supplemental oxygen.

According to some embodiments, the types of facial devices include any one or more of a nasal cannula, an oral-nasal oxygen mask, a non-rebreather mask, high flow nasal cannula, high flow oxygen delivery devices, and a full face oxygen mask.

According to some embodiments, the treatment parameters include the delivery mode of the supplemental oxygen, and wherein the treatment parameters further specify, per each physical delivery mode, one or more types of facial devices compatible with the delivery mode.

According to some embodiments, the controller includes a processor and a memory, which stores software instructions executable by the processor, the software instructions including one or more algorithms, which are derived using machine learning tools and/or data mining tools, and wherein the analysis performed by the controller includes applying the one or more algorithms.

According to some embodiments, the one or more algorithms include an artificial neural network and/or a decision tree.

According to some embodiments, the communication infrastructure is configured to communicatively associate the controller with a hospital computer and/or an online server.

According to some embodiments, the communication infrastructure includes a wireless communication unit.

According to some embodiments, the controller is configured to relay, via the communication infrastructure, the values of the sensor-obtained respiratory parameters and the treatment parameters to the hospital computer and/or the online server.

According to some embodiments, the controller is configured to receive, via the communication infrastructure, updates to the one or more algorithms from the hospital computer and/or the online server.

According to some embodiments, the controller is configured to relegate to the hospital computer and/or the online server some or all of the computations, involved in analyzing the sensor-obtained respiratory parameters of the subject, when communication therewith is present and stable.

According to some embodiments, the sensors include one or more of a pulse oximeter, respiratory rate, a heart-rate sensor, a FiO₂ sensor, a capnograph, an ECG sensor, a blood pressure sensor, a thermometer, a galvanic skin response sensor, a motion sensor, an activity sensor and a pedometer.

According to some embodiments, the subject specific data include one or more of a gender, age, weight, ethnicity, and one or more medical conditions of the subject.

According to some embodiments, the subject specific data further includes one or more of at least a partial medical history of the subject and a response of the subject to past treatments.

According to some embodiments, the medical subgroup of subjects is characterized by a same gender, age bracket, ethnicity and/or weight category as the subject and/or are diagnosed with a same or a similar medical condition as the subject.

According to some embodiments, the system may be configured for treatment of one or more of a respiratory infection, asthma, chronic obstructive pulmonary disease, and cystic fibrosis.

According to some embodiments, the respiratory infection is viral and/or bacterial and/or wherein the respiratory infection is manifested at least as pneumonia.

According to some embodiments, the respiratory infection is corona and/or tuberculosis.

According to some embodiments, the system includes the console, and wherein the console includes a user interface, which is functionally associated with the controller and configured to allow a caregiver to set the treatment parameters.

According to some embodiments, the communication infrastructure is configured to associate the controller with a monitor, so as to allow displaying on the monitor one or more of the monitored respiratory treatment parameters, one or more of the treatment parameters and/or one or more of the recommended treatment parameters, and, optionally, subject specific data.

According to some embodiments, the monitor is a touch screen, and the controller is configured to be controlled via the touch screen.

According to some embodiments, the controller may further be configured to define normal or predefined ranges of the supplemented oxygen flow rate and/or the fraction of inspired oxygen (FiO₂), monitor a trend in the supplemented oxygen flow rate and/or the fraction of inspired oxygen (FiO₂) during treatment, and if a persistent trend (increase or decrease) is detected, activate a respective alarm indicative of a deterioration or an improvement in the subject's condition.

According to some embodiments, the controller may further be configured to provide a recommendation to the subject, the recommendation comprises: a number of hours per day the subject is required to receive supplemental oxygen treatment, a duration of a period in which the subject can take a break from supplemental oxygen treatment and/or from one or more sensors (of course, the subject may choose to reconnect to the supplemental oxygen treatment/sensor(s) should they experience a deterioration), indication of a time, when the subject is required to provide physiological parameters, or any combination thereof.

According to some embodiments, the controller may further be configured to assess the subject's physical activity, for example, utilizing a motion sensor, activity sensor and/or pedometer. Depending on the results of the assessment, the controller may suggest a consciousness level assessment. Depression of consciousness may suggest CO₂ narcosis. Advantageously, physical activity assessment may assist in determining when hypoxemia is real as opposed to an artifact due to patient movement or weak pulse oximeter signal. Moreover, according to some embodiments, if the system detects that the subject engages in physical activity (e.g., sports, climbing stairs, walking, etc.), the controller may further be configured to recommend or instruct automatically providing of more O₂ to prevent exertional hypoxemia.

According to some embodiments, the controller may further be configured to recommend a “challenge test”, such as a few (e.g., 3-6) minutes walk or climbing stairs, and predicting an upcoming oxygen desaturation, based at least on a result of the challenge test.

According to some embodiments, the system may be distributed in hospitals, homes, ambulances (EMS vehicles), rehab/skilled nursing facilities or in any other facility. According to additional or alternative embodiments, each patient may have their own system, which transfers with them throughout the continuum of care, and has their information, relevant disease, and oxygenation history.

According to additional or alternative embodiments, the system data for each individual patient, may be saved in a wearable device.

According to additional or alternative embodiments, one or more of the sensors may be in a form of a wearable device. Advantageously, a wearable device may be configured for one or more of the following objectives- to sense various parameters, to store data, to connect to a control device in the various care locations (home, ambulance, hospital, nursing home, etc.), and to provide alert(s) if certain parameters passed a threshold.

According to additional or alternative embodiments, the system data for each individual patient, may be saved in remote server/cloud.

According to some embodiments, sensors (e.g., miniature sensors) may be attached to the oxygen delivery device.

According to some embodiments the terms “patient” and “subject” may be used interchangeably.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIG. 1 shows a schematic illustration of a system for supplementary oxygen delivery, in accordance with some embodiments of the present invention; and

FIG. 2, which shows a flowchart of functional steps in a method for supplementary oxygen delivery, in accordance with some embodiments of the present invention.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there is provided a method and system configured to ensure safe and effective treatment using oxygen supplementation. According to some embodiments, the method and system as provided herein are configured to assist in controlling, documenting, analyzing, displaying and/or automating oxygen supplementation. According to some embodiments, the method and system are configured to assist caregivers in making changes to various parameters, as described in greater detail elsewhere herein.

According to some embodiments, there is provided a method and system configured to provide comprehensive solutions to control and optimize supplemental oxygen delivery and to assist in clinical decision making in real time.

According to some embodiments, the system may include a console in communication with a oxygen outflow valve that may digitize oxygen flow. According to some embodiments, the system may include inlets configured to receive input from sensors and/or monitors. According to some embodiments, the sensors and/or monitors may be configured to measure SaO₂, respiratory rate, heart rate and/or additional physiological parameters such as blood O₂, blood CO₂, exhaled CO₂ and oxygen reserve index (ORI). According to some embodiments, the system may be configured to control oxygen flow automatically, such as, for example, based on closed loop feedback or by a push of a button, thereby being able to determine flow rate.

According to some embodiments, the system may include one or more inlets configured to couple with one or more oxygen delivery device such as a cannula or a mask. According to some embodiments, the one or more inlets may include one or more sensors, labels, and/or chips configured to identify the oxygen delivery device. Advantageously, identifying the oxygen delivery device may enable the system to document, save and/or present data associated with the oxygen delivery device or the oxygen supplementation treatment.

According to some embodiments, the system may be configured to control oxygen delivery as well as assist in clinical decision making by presenting comprehensive, accurate and complete data associated with the treatment of the subject. According to some embodiments, the system may be configured to communicate with an electronic medical record (EMR) system or a digital health record. According to some embodiments, the system may be configured to provide data on physiological parameters of the subject.

According to some embodiments, the system may be configured to diagnose that suboptimal oxygen delivery is being provided to a subject. According to some embodiments, the system may be configured to identify a currently used oxygen delivery device and/or mode as a suboptimal oxygen delivery device and/or mode.

According to some embodiments, the system may include an algorithm configured to display data associated with the oxygen delivery device or the oxygen supplementation treatment. According to some embodiments, the system may include a display, such as a digital display. According to some embodiments, the display may be a display of an external computer, i.e., that may not be part of the system.

According to some embodiments, the method may include displaying the physiological parameters over time, such as within at least 24 to 48 hours. According to some embodiments, the method may include displaying the time at which changes in oxygen delivery were made, such as, for example, a change of the oxygen delivery device, change in oxygen percent, change in the oxygen flow rate, or any combination thereof.

Advantageously, enabling the caregiver to observe and analyze trends in oxygen delivery and outcomes on SaO₂ and/or respiratory rate increases the ability of the caregiver in making clinical decisions, such as changing treatment, adding a treatment, and the like.

According to some embodiments, the system and methods may include a feedback mechanism involving a feedback loop of the detected physiological parameters to a digital system. According to some embodiments, the feedback mechanism may be configured to assess the physiological parameters, for example, in real time, and may be configured to suggest changes in the treatment of the subject.

Commonly, a subject can initially have acute respiratory failure with a high respiratory rate but normal SaO₂, as the fast respiration at the initial phase of disease can compensate such that SaO₂ is maintained at a normal level, but as the subject becomes exhausted from the breathing effort, quick desaturation is common to occur. Therefore, identifying a high respiratory rate (with or without a fast heart rate) may indicate an impending risk for desaturation and may determine the need for clinical intervention.

According to some embodiments, the method and system as disclosed herein enable utilization and implementation of machine learning algorithms. According to some embodiments, the machine learning algorithms may be trained using a training set that may include a database having a plurality of subjects and the medical histories of the plurality of subjects. According to some embodiments, the algorithm may be configured to identify a medical subgroup that is the same or similar to the treated subject. According to some embodiments, the algorithm may be configured to update the database in real time.

According to some embodiments, the medical subgroup may include subjects which had similar conditions and/or are undergoing similar treatment as the treated subject. According to some embodiments, the medical subgroup may be characterized by a same gender, age bracket, ethnicity and/or weight category as the subject and/or are diagnosed with a same or a similar medical condition as the treated subject. According to some embodiments, the medical subgroup may include subjects suffering from similar medical comorbidities, such as cardiac and respiratory conditions, and/or subjects that are receiving similar treatments for their chronic conditions.

According to some embodiments, the algorithm may be configured to incorporate multiple parameters such as age, sex, chronic conditions, acute disease, saturation, respiratory rate and other parameters to assist with determining target SaO₂ level. For example, the target SaO₂ for subjects suffering from chronic obstructive pulmonary disease (COPD) is often 88-92% whereas a target of 94-98% is usually the case for acutely ill subjects not at risk of hypercapnic respiratory failure. According to some embodiments, the algorithm may be configured to predict risk for further deterioration of the health of the treated subject. According to some embodiments, the algorithm may be configured to output recommendations for the caregiver, wherein the recommendations may be associated with changes in oxygen delivery.

According to some embodiments, the algorithm may be configured to adjust the treatment based on the baseline health of the subject and/or based on the medical history of the subject. According to some embodiments, the algorithm may be configured to adjust the treatment based on whether the subject is healthy or has COPD. According to some embodiments, the algorithm may be configured to adjust the treatment based on age, general health, and functional status of the treated subject. According to some embodiments, the algorithm may be configured to adjust the treatment based on the type of condition that is being treated, such as, an account acute condition or chronic condition.

According to some embodiments, the algorithm may be configured to automate changes and/or to present predictive analytics which would guide clinical decision making. According to some embodiments, the algorithm may be configured to suggest an increase or decrease of the O₂ flow, the FiO₂, and/or to change the oxygen delivery device. For example, if a subject with COPD is treated using a nasal cannula and the SaO₂ of the treated subject drops to 86%, (for example, below 88%, which may be defined as the minimal target SaO₂) on 3 L/min O₂, the algorithm may output one or more recommendations which may include: a suggestion to change to a mask with 5 L/min O₂, or a suggestion to raise flow to 5 L/min and remain on nasal cannula.

According to some embodiments, the algorithm may base the recommended treatment on personalized data of the treated subject. According to some embodiments, the algorithm may include subject-specific analyses which may include assessments of reactions of the treated subject to various changes and/or treatments. According to some embodiments, the algorithm may be configured to update the subject-specific analyses in real time. According to some embodiments, the algorithm may be configured to assess the basic status of the treated subject. According to some embodiments, basic status may include the duration of oxygen supplementation and/or status on room air (e.g., if oxygen delivery is stopped intermittently).

According to some embodiments, the algorithm may be configured to assess the response of the subject to oxygen treatment, such as at a response to adjustment of treatment, a change in treatment, throughout the duration of the treatment, and/or during periods between treatments. According to some embodiments, the response to adjustments could include the prior results (the impact of change on the subject related parameters) achieved due to change in oxygen flow rate, changes in FiO₂ or adjusting the method oxygen is delivered (impacts the FiO₂ and CO₂ in inhaled air).

Advantageously, assessing the response of the subject increases the ability to understand and predict which adjustments would optimize care (or treatment) as the subject is deteriorating or improving. Furthermore, according to some embodiments, the system may be configured to capture, analyze, and suggest current treatment options based on subject specific data from the current admission or recent (previous) admissions of the subject.

According to some embodiments, the system may be configured to assist with identifying, documenting and presenting the delivery device in use and may be configured to guide the caregiver in determining the need to change the delivery device. According to some embodiments, the delivery device may include any one or more of a nasal cannula, and various types of masks such as, venti-mask, non-rebreather, and the like, and any combination thereof.

According to some embodiments, the system may be configured to provide digital data associated with the type of delivery device that is in use. According to some embodiments, the system may be configured to provide data associated with the correlation between the type of delivery device and the physiological parameters of the subject. Advantageously, providing digital data associated with the type of delivery device that is in use removes the need of a manual documentation of a caregiver which may be lacking or inaccurate. According to some embodiments, the system may be configured to document the type of delivery device and/or the oxygen delivery mode. According to some embodiments, the system may be configured to document the type of delivery device and/or the oxygen delivery mode using one or more of a user interface, automatic detection, and/or image recognition.

According to some embodiments, the system may be configured to document the type of delivery device and/or the oxygen delivery mode using a user interface which may include a touch screen and/or one or more buttons. According to some embodiments, the user interface may include a plurality of delivery device options for a user to select from. According to some embodiments, the system is configured to transmit the selected data from the user interface to the EMR. According to some embodiments, once the delivery device is selected on the user interface, data associated with the type of deliver device is transmitted to the EMR.

According to some embodiments, the automatic detection may include a mechanism for automatically detecting a delivery device that may be coupled to the system. According to some embodiments, the delivery device may be coupled to a digital console and may include a chip and/or label that may be detected within the system and wherein the chip and/or label may be associated with a type of delivery device. According to some embodiments, the system may include a sensor configured to detect the chip or label of the delivery device. According to some embodiments, the chip may include and/or produce an electronic signal associated with a type of delivery device. According to some embodiments, the label may include a barcode associated with a type of delivery device. Advantageously, a system which includes a chip and/or label that may be detected and associated with a type of delivery device enables the system to automatically identify the type of delivery device that is being used.

According to some embodiments, the image recognition mechanism may include a camera in communication with a controller, wherein the controller may be configured to identify the type of delivery device (such as, nasal cannula, type of mask, and the like), based on image data received from the camera. According to some embodiments, the image recognition mechanism and/or the controller may be configured to identify a dislocated delivery device (such as, for example, a falling mask or cannula) and may be configured to output an alert about misplacement of the delivery device.

According to some embodiments, the system may include a sensor, such as a barometer or a micro-barometer in communication with the controller. According to some embodiments, the controller may be configured to detect misplacement and/or removal of the deliver device using data received from the sensor. According to some embodiments, the controller may be configured to detect a change in resistance associated with change in the placement of the delivery device placed on the subject. For example, the controller may be able to detect significant amounts of oxygen that may be leaking from the sides of a mask positioned on the subject, using data received from the sensor.

According to some embodiments, the system may be configured to display comprehensive data on a digital console (such as, for example, a screen or display) as well as transmit the comprehensive data to the EMR. According to some embodiments, the comprehensive data may include one or more tables with at least one of the physiological parameters (such as, for example, the respiratory rate and/or the oxygen saturation). According to some embodiments, the comprehensive data may include one or more tables with at least one of the physiological parameters as a function of time. According to some embodiments, the comprehensive data may include indication of changes that were made to O₂ flow and/or a change of the delivery device. According to some embodiments, the comprehensive data may include graphs of one or more physiological parameters, such as respiratory rate and/or oxygen saturation (SaO₂) as a function of time. According to some embodiments, the system may be configured such that the user can define how often the comprehensive data will be captured on the screen, for example, if the presented comprehensive data is continuously presented or is presented at small or large time intervals. According to some embodiments, the duration of the presentation of the comprehensive data on the display or screen may be based on preference and/or the stability of the subject. According to some embodiments, the duration of the presentation of the comprehensive data on the display or screen may be automatic, such that the less stable a subject, the more data points that will be presented, thereby enabling the caregiver to receive a better understanding of clinical status of the subject.

According to some embodiments, the system may include an option to allow subject-controlled oxygen delivery following enabling of this function by the caregiver. According to some embodiments, the subject may be able to adjust oxygen flow, such as to increase or decrease within a default spectrum. According to some embodiments, the subject may be able to adjust oxygen flow within a predefined and/or subject-specific spectrum determined by the caregiver. According to some embodiments, the subject may be able to adjust oxygen flow within a predefined and/or subject-specific spectrum determined by the algorithm.

According to some embodiments, the system and/or user interface may include a pause mechanism in which a user and/or caregiver may select a pause in treatment and/or a pause in usage of the oxygen delivery device. According to some embodiments, the pause mechanism may be used while nasal cannula or mask are being removed whether for examining the subject, cleaning/changing mask or as the subject is eating/drinking. Advantageously, indicating of the times in which the oxygen delivery device is removed and/or the treatment is paused enables the system to understand the associated intermittent fall in SaO₂ and/or prevent unnecessary alarms. According to some embodiments, a fall in SaO₂ associated with a pause in treatment will not disable the SaO₂ alarm from ringing once it goes below the alarm threshold. According to some embodiments, the system may include an option to allow the subject to press pause while eating/drinking or using the restroom, for example, for subjects who are stable or improving and/or subject that may have one or more physiological parameters above a predetermined threshold for a predetermined period of time.

According to some embodiments, the system may include a console for humidity and/or temperature control. According to some embodiments, the console may be coupled to the oxygen control device to further optimize the comfort of the subject, as well as increase the compliance and positively impact the outcome of the oxygen treatment. According to some embodiments, the console for humidity and/or temperature control may include one or more of a nebulizer, vaporizer or humidifier, or any combination thereof.

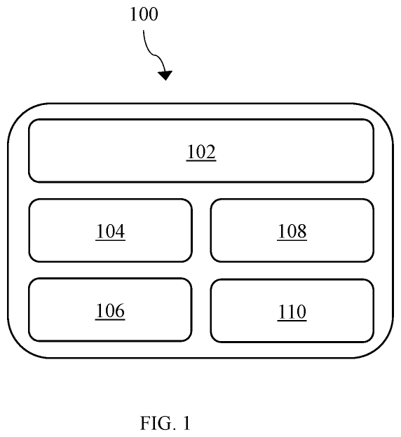

Reference is made to FIG. 1 shows a schematic illustration of a system for supplementary oxygen delivery, in accordance with some embodiments of the present invention.

According to some embodiments, there is provided a system 100 for supplementary oxygen delivery. According to some embodiments, the system 100 may be configured for treatment of one or more of a respiratory infection, asthma, chronic obstructive pulmonary disease, and cystic fibrosis. According to some embodiments, the respiratory infection may include a viral infection, bacterial infection, mycobacterium, fungal, and/or aspiration pneumonia. According to some embodiments, the respiratory infection may be manifested at least as pneumonia. According to some embodiments, the respiratory infection may include covid-19, pneumococcal, and/or tuberculosis.

According to some embodiments, the system may include at least one hardware processor, such as controller 102. According to some embodiments, the system 100 may include a memory module 104 in communication with the controller 102. According to some embodiments, the memory module 104 may include a non-transitory computer-readable storage medium having stored thereon program code. According to some embodiments, the program code may be executable by the at least one hardware processor and/or controller 102 to obtain data associated with an oxygen treatment of the subject, as described in greater detail elsewhere herein. According to some embodiments, the controller 102 may be in communication with the electronic medical record (EMR) system or digital health record.

According to some embodiments, the system 100 may be portable and/or configured to home use. According to some embodiments, the system 100 may be configured to couple to one or more oxygen tanks and/or to oxygen concentrators. According to some embodiments, and as described in greater detail elsewhere herein, the system 100 may be configured to communicate with one or more memory modules, such as memory module 104, that may be used for any one or more of remote home health monitoring, caregiver remote monitoring, telemedicine and/or telemonitoring platforms. According to some embodiments, memory module 104 may include a remove storage unit, such as a cloud.

According to some embodiments, the system 100 may include a controller 102 and a communication infrastructure configured to communicatively associate the controller 102 with an electronic valve of a console 106, which is fluidly coupled to an oxygen source and configured to control a flow of supplemental oxygen, and a plurality of sensors 108 configured to monitor physiological parameters of the subject. According to some embodiments, the communication infrastructure may be configured to communicatively associate the controller 102 with a hospital computer and/or an online server. According to some embodiments, the communication infrastructure may include a wireless communication unit. According to some embodiments, the controller 102 may be configured to relay, via the communication infrastructure, the values of the sensor-obtained physiological parameters and/or one or more treatment parameters to the hospital computer and/or the online server. According to some embodiments, the controller 102 may be configured to relegate to the hospital computer and/or the online server some or all of the computations, involved in analyzing the sensor-obtained physiological parameters of the subject, when communication therewith is present and stable.

According to some embodiments, the memory module 104 may include a database of subjects that have received oxygen supplemental treatments. According to some embodiments, the memory module 104 may be configured to store data associated with the subject and/or the treatment of the subject, such as the physiological parameters of the subject over time, the medical history of the subject, clinical data of the subject, demographic data of the subject, and the like. According to some embodiments, the physiological parameters may include any one or more of oxygen saturation (SaO₂), heart rate, respiratory rate, blood O₂ level, blood CO₂ level, exhaled CO₂ level, and oxygen reserve index. According to some embodiments, the physiological parameters may include any one or more of a temperature, blood pressure, and/or blood sugar level of the treated subject.

According to some embodiments, the medical history of the subject may include any one or more of inputted medical history of the subject, data received from the EMR, previous treatments such as previous treatments using oxygen supplementations, physiological parameters of the subject during and/or after previous treatments using oxygen supplementations, and the like. According to some embodiments, the clinical data of the subject may include data associated with medical conditions, such as acute conditions and/or chronic conditions of the subject. According to some embodiments, the demographic data of the subject may include any one or more of the gender, age bracket, ethnicity and/or weight category of the subject.

According to some embodiments, medical conditions of the subject may include, but are not limited to respiratory infection, asthma, emphysema, chronic obstructive pulmonary disease, pulmonary hypertension, pulmonary fibrosis and cystic fibrosis, congestive heart failure, myocardial infarction, cardiac arrest, sepsis, stroke, trauma, and the like.

According to some embodiments, the controller 102 may be configured to receive, via the communication infrastructure, updates to one or more algorithms of the memory module 104, from the hospital computer and/or the online server. According to some embodiments, and as described in greater detail elsewhere herein, the memory module 104 may include a program code having instructions configured to execute the method for supplementary oxygen delivery, such as, for example, method 200 described hereinbelow. According to some embodiments, the memory module 104 may include a program code including software instructions executable by the controller 102. According to some embodiments, the software instructions may include one or more algorithms. According to some embodiments, the one or more algorithms may include machine learning algorithms and/or may be derived using machine learning tools and/or data mining tools. According to some embodiments, the one or more algorithms may include one or more artificial neural networks and/or decision trees.

According to some embodiments, the program code may include one or more algorithm, such as a machine learning algorithm, configured to generate instructions to be implemented by the system 100 and/or controller 102, such that method 200 is executed by the system 100 and/or controller 102. According to some embodiments, the one or more algorithm may be trained on a training set including one or more databases including a plurality of subjects of the population data. According to some embodiments, the one or more algorithm is configured to learn the medical history of the treated subject in real time. According to some embodiments, the one or more algorithm is configured to identify a medical subgroup similar to the treated subject in real time. According to some embodiments, the one or more algorithms may be configured to assess the optimal treatment parameters for the treated subject. According to some embodiments, the one or more algorithm may be configured to asses which modifications, if any, should be made to the treatment parameters of the treatment of the subject.

According to some embodiments, the system 100 may include a console 106 which may include one or more oxygen delivery devices. According to some embodiments, the one or more oxygen delivers devices may include any one or more of a nasal cannula, an oral-nasal oxygen mask, a non-rebreather mask, and a full face oxygen mask, nasal progs, tracheostomy mask, venturi mask, a high flow nasal cannula (HFNC), or other high flow devices, and/or any combination thereof. According to some embodiments, the console 106 may be in operative communication with an oxygen outflow valve, such as an electronic valve or a mechanical valve configured to regulate the flow of oxygen to the oxygen delivery device. According to some embodiments, the console 106 may include an inlet port and an outlet port, each of which may be fluidly-coupled to the valve. According to some embodiments, the inlet port may be couplable to a first tube so as to fluidly couple the valve to the oxygen source, and the outlet port may be couplable to a second tube so as to fluidly couple the valve to a delivery device, or facial device, for supplemental oxygen delivery.

According to some embodiments, the system 100 may include one or more sensors 108 associated with the console 106 and/or with the oxygen delivery device. According to some embodiments, the one or more sensors may be configured to be positioned onto and/or adjacent to the treated subject. According to some embodiments, the one or more sensors may be configured to detect one or more physiological parameters of the treated subject. According to additional or alternative embodiments, the one or more sensors may be configured to detect one or more treatment parameters. According to some embodiments, and as described in greater detail herein, the one or more sensors may be configured to detect a type of oxygen delivery device that is coupled to the console 106. According to some embodiments, the one or more sensors may be configured to detect a chip or label associated with the oxygen delivery device.

According to some embodiments, the one or more sensors may include any one or more of a pulse oximeter, respiratory rate monitor, a heart-rate sensor, a FiO₂ sensor, a capnograph, an ECG sensor, a blood pressure sensor, a thermometer, a galvanic skin response sensor, a motion sensor, an optical sensor, a microphone or audio sensor, a camera and/or video camera, a pressure sensor, a humidity sensor, a barometer, a micro-barometer, an IR sensor, thermal sensor, and any combination thereof.

For example, according to some embodiments, a camera and/or video camera may be used to visually track the breath of the subject. For example, according to some embodiments, the controller 102 may be configured to track the breath of the subject using data received from the camera and/or video camera.

According to some embodiments, the one or more treatment parameters may include a supplemented oxygen flow rate, a fraction of inspired oxygen, a type of oxygen delivery device that is used, a duration of treatment, a duration of time between intervals of treatment, intervals of oxygen supplementation, timing of oxygen supplementation, and the like. According to some embodiments, the one or more treatment parameters may include a delivery mode of the supplemental oxygen, which may include a type of oxygen delivery device and a supplemented oxygen flow rate.

According to some embodiments, the sensors 108 may be configured to measure and/or detect one or more of a saturation (SaO₂), respiratory rate, heart rate, blood O₂, blood CO₂, volume of breath, exhaled CO₂ and oxygen reserve index (ORI) of the subject, activity of the subject, steps of the subject or any combination thereof. According to some embodiments, the sensors 108 may be configured to measure and/or detect one or more parameters obtained by performing one or more pulmonary function tests, such as, but not limited to, tidal volume and total lung capacity. Each possibility is a separate embodiment.

According to some embodiments, the system 100 may include a user interface 110, which may be functionally associated with the controller 102. According to some embodiments, the user interface 110 may be configured to allow a caregiver to set one or more of the treatment parameters. According to some embodiments, the user interface 110 may include a touch screen and/or one or more buttons, a keyboard, joystick, and the like. According to some embodiments, the system 100 and/or the user interface 110 may include one or more displays configured to present a user and/or caregiver with data associated with the treated subject and/or the treatment of the subject. According to some embodiments, the user interface 110 may include speakers and/or one or more audio delivery devices configured to audibly communicate and/or alarm a subject or caregiver.

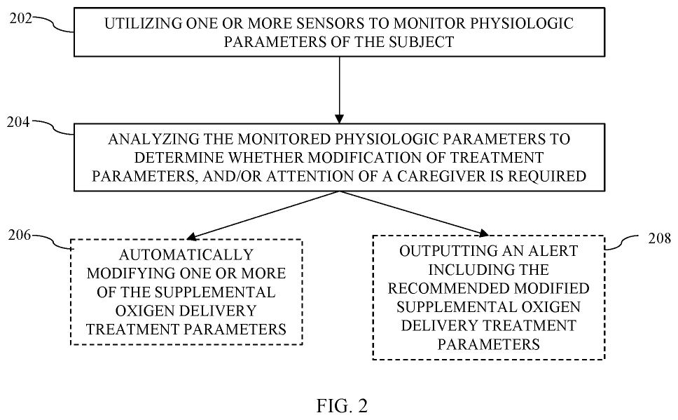

Reference is made to FIG. 2, which shows a flowchart of functional steps in a method for supplementary oxygen delivery, in accordance with some embodiments of the present invention.

According to some embodiments, the method 200 may be a computerized method, or in other words, may be configured to be implemented by a processor or controller, such as controller 102. According to some embodiments, the method 200 may be integral with one or more instructions stored onto memory module 104 of system 100. According to some embodiments, controller 102 and/or system 100 may be configured to implement the method 200. According to some embodiments, the method 200 may be configured for monitoring a subject that is receiving supplemental oxygen. According to some embodiments, the method 200 may be configured for controlling supplemental oxygen delivery. According to some embodiments, the method 200 may be configured for recommending a change associated with the supplemental oxygen delivery treatment.

According to some embodiments, at step 202, the method may include utilizing one or more sensors to monitor physiologic parameters of the subject. According to some embodiments, at step 204, the method may include analyzing the monitored physiologic parameters to determine whether modification of treatment parameters, and/or attention of a caregiver is required. According to some embodiments, the method may include identifying a current oxygen deliver device and/or mode as a suboptimal oxygen delivery device and/or mode.

According to some embodiments, if modification of treatment parameters is assessed to be required, at step 206, the method may include automatically modifying one or more of the treatment parameters. According to some embodiments, if attention of a caregiver is assessed to be required, at step 208, the method may include outputting an alert including the recommended modified treatment parameters.

According to some embodiments, the method may include collecting data associated with the treated subject. According to some embodiments, the data associated with the treated subject may be inputted by a user, the treated subject, and/or a caregiver. According to some embodiments, the data associated with the treated subject may include a medical history of the treated subject, clinical data of the treated subject, and/or demographic data of the treated subject. According to some embodiments, the data associated with the subject may include one or more physiological parameters of the treated subject. According to some embodiments, the method may include detecting, using one or more sensors, such as, for example, the one or more sensors 108 of system 100, the one or more physiological parameters of the treated subject.

According to some embodiments, the method may include assessing a baseline health of the subject based on any one or more of the medical history, clinical data, demographic data, and/or initial physiological parameters of the treated subject. According to some embodiments, the method may include assessing a baseline health of the subject based, at least in part, on data stored onto the database which may be associated with a population data, such as, for example, the data from which the medical subgroup is formed.

According to some embodiments, the method may include identifying and/or forming the medical subgroup associated with the treatment of the treated subject, or in other words, the method may include generating a medical subgroup from the population data of the database, wherein the medical subgroup may include a plurality of subjects which have previously received a same or similar treatment as the treated subject. According to some embodiments, the medical subgroup may be based on a same acute condition, a same chronic condition and/or similar diagnoses of the treated subject and the plurality of subjects selected into the medical subgroup. According to some embodiments, the method may include generating a subgroup including a plurality of subjects that are receiving or have previously received supplemental oxygen. According to some embodiments, the method may include generating the medical subgroup based on any one or more of a same gender, age bracket, ethnicity and/or weight category as the subject and/or are diagnosed with a same or a similar medical condition as the subject. According to some embodiments, the method may include generating the medical subgroup based on subjects that are and/or were suffering from similar medical comorbidities, such as cardiac and respiratory conditions, and/or subjects that are receiving and/or were receiving similar treatments for their chronic conditions.

According to some embodiments, the method may include analyzing the medical records and/or previous treatments of the treated subject. According to some embodiments, the method may include identifying causes for deterioration and/or improvements of previous treatments of the treated subject.

According to some embodiments, at step 202, the method may include utilizing one or more sensors to monitor physiologic parameters of the subject. According to some embodiments, the method may include receiving data from one or more sensors to monitor physiologic parameters of the subject. According to some embodiments, the method may include monitoring physiologic parameters using the one or more sensors.

According to some embodiments, the method may include monitoring the physiologic parameters continuously or semi-continuously. According to some embodiments, the method may include receiving input, from a caregiver, associated with a rate of monitoring the physiologic parameters of the subject. According to some embodiments, the method may include calculating a rate of monitoring the physiologic parameters of the subject, wherein an increase in severity of the health of the subject may initiate an increase in the rate of monitoring of the physiologic parameters of the subject. According to some embodiments, the method may include monitoring the physiologic parameters of the subject continuously. According to some embodiments, the method may include monitoring the physiologic parameters of the subject at a rate of at least every minute, every 10 minutes, and every 15 minutes.

According to some embodiments, the method may include monitoring the physiologic parameters at different intervals in different times (such as, for example, different times of the day and/or specific times associated with other treatments that the subject is receiving). According to some embodiments, the system may be configured to monitor the physiological parameters at different intervals at different times. For example, if a subject desaturates more during sleep, then monitoring may be continuous, or every minute, however, during the day, the rate of monitoring the same subject may be reduced to every 10-15 minutes. According to some embodiments, the algorithm of the system may be configured to identify different times of the day which may require different monitoring rates of the physiological parameters of the subject. According to some embodiments, the algorithm may be configured to associate different rates of monitoring of the physiological parameters to different times of the day and/or times associated with other treatments that the subject is receiving.

According to some embodiments, the method may include identifying and/or detecting a type of oxygen delivery device used to treat the subject. According to some embodiments, the method may include receiving input, from a user or caregiver, associated with a type of oxygen delivery device used to treat the subject. According to some embodiments, the method may include detecting a chip or label, such as a barcode, positioned onto the oxygen delivery device. According to some embodiments, the method may include identifying the oxygen delivery device using image processing, based on data received from a camera or video camera positioned towards the treated subject.

According to some embodiments, at step 204, the method may include analyzing the monitored physiologic parameters to determine whether modification of treatment parameters, and/or attention of a caregiver is required. According to some embodiments, the method may include comparing the monitored physiologic parameters to analyzed medical records of the treated subject. According to some embodiments, the method may include comparing the monitored physiologic parameters to the population data and/or the generated medical subgroup. According to some embodiments, the method may include comparing the monitored physiologic parameters to the subgroup including the plurality of subjects that are receiving or have previously received supplemental oxygen.

According to some embodiments, the method may include calculating a target SaO₂ level. According to some embodiments, the method may include calculating a target SaO₂ level based, at least in part, on the clinical data, medical history, and/or demographic data of the treated subject. According to some embodiments, the method may include calculating a target SaO₂ level based, at least in part, on data associated with the medical subgroup.

According to some embodiments, the method may include measuring the SaO₂ level of the treated subject. According to some embodiments, for a measured SaO₂ level that is lower than the calculated target SaO₂ level, the method may include assessing if the SaO₂ level will increase under the current treatment to sufficiently near the target SaO₂ level. According to some embodiments, for a measured SaO₂ level that is lower than the calculated target SaO₂ level, and when the SaO₂ level is assessed not to increase under the current treatment to sufficiently near the target SaO₂ level, the method may include calculating adjusted values of the treatment parameters, which may be configured to raise the measured SaO₂ level to the target SaO₂ level.

According to some embodiments, if modification of treatment parameters is assessed to be required, at step 206, the method may include automatically modifying one or more of the treatment parameters. According to some embodiments, the method may include automatically modifying one or more of the treatment parameters by controlling the valve of the console.

According to some embodiments, if attention of a caregiver is assessed to be required, at step 208, the method may include outputting an alert including the recommended modified treatment parameters. According to some embodiments, the method may include alerting the caregiver and/or subject, and/or providing thereto recommended modified supplemental oxygen delivery treatment parameters. According to some embodiments, the method may include outputting one or more of an audio alarm, such as a bell. According to some embodiments, the method may include outputting voice instructions of the recommended modified treatment parameters and/or voice instructions including recommendations for adjustments which may result in the desired modified treatment parameters. According to some embodiments, the method may include outputting a message onto the display which may include the recommended modified treatment parameters and/or recommendations for adjustments which may result in the desired modified treatment parameters.

According to some embodiments, the automatically modifiable treatment parameters may be limited to modifications to within a treatment range, and modifications to outside the treatment range require intervention of caregiver. According to some embodiments, the automatically modifiable treatment parameters may be limited to published guidelines provided by respectable committees, conferences, groups, which may be incorporated into the one or more algorithms. According to some embodiments, the recommended values of at least some of the treatment parameters may be limited to within a respective safety range, as well as published guidelines provided by respectable committees, conferences, groups, and the like.

According to some embodiments, the automatically modifiable treatment parameters and/or the recommended modified treatment parameters may include one or more one or more types of facial devices, or delivery devices, compatible with the delivery mode. According to some embodiments, the automatically modifiable treatment parameters and/or the recommended modified treatment parameters may include a range of flow rate of oxygen.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1. A computerized method for monitoring a subject receiving supplemental oxygen and/or being in risk of requiring supplemental oxygen and controlling supplemental oxygen delivery thereto, the method comprising operations of:
utilizing one or more sensors to monitor physiologic parameters of the subject, the physiologic parameters comprising at least oxygen saturation (SaO2) level and one or more of respiratory rate and heart rate;
utilizing a processing circuitry to analyze the monitored physiologic parameters, and subject specific data and/or population data, to determine whether (i) modification of supplemental oxygen delivery treatment parameters/initiation of treatment with supplemental oxygen, and/or (ii) attention of a caregiver, is required; and
if at least (i) is required:
automatically modifying one or more of the supplemental oxygen delivery treatment parameters; and
if at least (ii) is required:
alerting the caregiver and/or subject, and/or providing thereto recommended modified supplemental oxygen delivery treatment parameters;
wherein the supplemental oxygen delivery treatment parameters comprise a supplemented oxygen flow rate, type of oxygen delivery device and/or a fraction of inspired oxygen (FiO₂); and
wherein the population data comprise treatment data of a plurality of subjects receiving supplemental oxygen and in a same medical subgroup as the subject.

2. The method of claim 1, wherein the analysis operation comprises:
(a) computing a target SaO₂ level; and
contingent on a current SaO₂ level being sufficiently lower than the target SaO₂ level, and, optionally, further assessed not to increase under the current treatment to sufficiently near the target SaO₂ level:
(b) computing adjusted values of the treatment parameters, which are configured to raise the current SaO₂ level to the target SaO₂ level.

3. The method of claim 2, wherein, contingent on (b), in the operation of modifying the treatment parameters, if implemented, the treatment parameters are modified to the adjusted values, and in the operation of providing the recommendation, if implemented, the adjusted values are recommended.

4. The method of claim 1, wherein one or more of the automatically modifiable treatment parameters are limited to modifications to within a treatment range, and modifications to outside the treatment range require intervention of caregiver.

5. The method of claim 4, wherein the treatment range of an automatically modifiable treatment parameter is population and/or subject-specific.

6. The method of claim 1, wherein the recommended values of at least some of the treatment parameters are limited to within a respective safety range.

7. The method of claim 1, wherein, in the monitoring operation, the physiologic parameters are monitored continuously or semi-continuously.

8. The method of claim 1, wherein, in the monitoring operation, the physiologic parameters are repeatedly sampled at a default rate or at a dynamic rate correlating with a level of severity of the condition of the subject.

9. (canceled)

10. (canceled)

11. (canceled)

12. (canceled)

13. The method of claim 1, wherein the subject is further monitored for one or more additional physiological parameters other than respiratory parameters;
wherein in the operation of analyzing the monitored respiratory parameters one or more additional physiological parameters are taken into account; and
wherein the one or more physiological parameters comprise a temperature, blood pressure, and/or blood sugar level of the subject.

14. The method of claim 13, wherein the respiratory infection is viral and/or bacterial and/or mycobacterium and/or fungal and/or aspiration pneumonia wherein the respiratory infection is manifested at least as pneumonia.

15. The method of claim 14, wherein the respiratory infection is COVID-19 and/or pneumococcal and/or tuberculosis.

16. (canceled)

17. (canceled)

18. The method of claim 1, wherein the treatment parameters further comprise intervals of oxygen supplementation and timing of oxygen supplementation.

19. (canceled)

20. (canceled)

21. (canceled)

22. (canceled)

23. (canceled)

24. The method of claim 1, further comprising defining normal or predefined ranges of the supplemented oxygen flow rate and/or the fraction of inspired oxygen (FiO₂), monitoring a trend in the supplemented oxygen flow rate and/or the fraction of inspired oxygen (FiO₂) during treatment, and if a persistent trend (increase or decrease) is detected, activating a respective alarm indicative of a deterioration or an improvement in the subject's condition.

25. The method of claim 1, further comprising providing a recommendation to the subject, the recommendation comprises: a number of hours per day the subject is required to receive supplemental oxygen treatment, a duration of a period in which the subject can take a break from supplemental oxygen treatment and/or from one or more sensors, indication of a time, when the subject is required to provide physiological parameters, or any combination thereof.

26. The method of claim 1, further comprising assessing the subject's physical activity.

27. The method of claim 1, further comprising recommending a challenge test and predicting an upcoming oxygen desaturation, based at least on a result of the challenge test.

28. A system for regulating delivery of supplemented oxygen to a subject receiving oxygen therapy and/or being in risk of requiring supplemental oxygen, the system comprising a controller and communication infrastructure configured to communicatively associate the controller with (i) an electronic valve, which is fluidly coupled to an oxygen source and configured to control a flow of supplemental oxygen, (ii) a plurality of sensors monitoring physiologic parameters of the subject, wherein the controller is configured to:
receive values of sensor-obtained physiologic parameters of the subject;
analyze said values and subject specific data and/or population data to determine whether (i) modification of supplemental oxygen delivery treatment parameters/initiation of treatment with supplemental oxygen, and/or (ii) attention of a caregiver and/or subject, is required; and
if at least (i) is required:
instruct the electronic valve to change the flow of supplemental oxygen, such as to modify one or more of the supplemental oxygen delivery treatment parameters; and
if at least (ii) required:
alert the caregiver and/or provide thereto recommended modified supplemental oxygen delivery treatment parameters;
wherein the physiologic parameters comprise at least oxygen saturation (SaO2) level and one or more of respiratory rate and heart rate;
wherein the supplemental oxygen delivery treatment parameters comprise a supplemented oxygen flow rate and a fraction of inspired oxygen; and
wherein the population data comprise treatment data of a plurality of subjects receiving supplemental oxygen and in a same medical subgroup as the subject.

29. The system of claim 28, wherein the analysis performed by the controller comprises:
(a) computing a target SaO₂ level; and
contingent on a current SaO₂ level being sufficiently lower than the target SaO2 level, and, optionally, further assessed not to increase under the current treatment to sufficiently near the target SaO₂ level:
(b) computing adjusted values of the treatment parameters, which are configured to raise the current SaO2 level to the target SaO2 level.

30. The system of claim 29, wherein, contingent on (b), and (i), the controller is configured to modify the one or more treatment parameters to the respective adjusted values; and
wherein, contingent on (b), and (ii), the controller is configured to recommend the adjusted values.

31. The system of claim 28, wherein the controller is configured such that one or more of treatment parameters automatically modifiable by the system are limited to modifications to within a treatment range, and modifications to outside the treatment range require authorization by a caregiver.

32.-61. (canceled)
